(19) United States
(12) Patent Application Publication
(10) Pub. No.: US 2023/0414911 A1
(43) Pub. Date: Dec. 28, 2023

(54) Intradermal Active Ingredient Delivery System

(71) Applicant: LTS Lohmann Therapie-Systeme AG, Andemach (DE)

(72) Inventor: Sebastian Schomisch, Mayen (DE)

(21) Appl. No.: 18/036,436

(22) PCT Filed: Oct. 27, 2021
(86) PCT No.: PCT/EP2021/079871

§ 371 Date: May 11, 2023

(30) Foreign Application Priority Data
Nov. 12, 2020 (DE) 10 2020 129 916.8

(51) Int. Cl. A61M 37/00; A61M 5/14

(52) U.S. Cl. CPC A61M 37/0015; A61M 2202/04; A61M 2037/0023; A61M 5/1407

(57) ABSTRACT

An intradermal active ingredient delivery system includes an extraction device, which in particular has a several hollow needles, for extracting interstitial liquid. A conveying device is fluidically connected to the extraction device. The conveying device is also fluidically connected to an active ingredient reservoir. The conveying device is fluidically connected to a delivery device for delivering an interstitial liquid comprising active ingredient. The delivery device preferably has hollow needles to be able to delivery the liquid mixture intradermally.

### CROSS-REFERENCE TO RELATED APPLICATIONS

This application is the United States national phase of International Application No. PCT/EP2021/079871 filed Oct. 27, 2021, and claims priority to German Patent Application No. 10 2020 129 916.8 filed Nov. 12, 2020, the disclosures of which are hereby incorporated by reference in their entireties.

### BACKGROUND OF THE INVENTION

### Field of the Invention

The invention relates to an intradermal active ingredient delivery system by means of which an active ingredient, in particular a medical active ingredient, can be delivered intradermally to a patient.

### Description of Related Art

Transdermal therapeutic systems (TTS) are known for active ingredient delivery. For example, transdermal patches can be applied to the patient's skin. The stored active ingredient is continuously resorbed through the skin. This form of administration has the advantage that, unlike injections, it is painless for the patient and, furthermore, medication can be administered over a longer period of time. However, a disadvantage is that medication adjustment can only be made by removing or replacing the TTS. In particular, it is difficult to clearly determine the amount delivered to the patient.

Furthermore, it is known to administer active ingredients via microneedles. Here, the active ingredient is delivered to the dermis of the skin via a plurality of needles. Thus, the delivery takes place in an outer layer of the skin in which no nerves are located, so that this active ingredient delivery is also painless for the patient. In this context, needles are known that are designed as hollow needles, in which active ingredient is contained and delivered through the needle opening at the needle tip. Moreover, microneedle are known that consist of a material that dissolves in the skin. Here, the active ingredient is incorporated into the needle material. Here, too, there is the problem that it is difficult to determine the exact amount of active ingredient delivered to the patient, since, for example, the needles do not dissolve completely, can break off, or the like.

### SUMMARY OF THE INVENTION

It is an object of the invention to provide an intradermal active ingredient delivery system that makes it easier to determine the amount of active ingredient delivered to the patient.

According to the invention, the object is achieved by an intradermal active ingredient delivery system having the features as described herein.

The intradermal active ingredient delivery system according to the invention comprises an extraction device for extracting interstitial liquid. Preferably, the extraction device has in particular several microneedles designed as hollow needles. A conveying device is fluidically connected to the extraction device. In particular, the conveying device is used to draw in the interstitial fluid from the patient's tissue. Moreover, the intradermal active ingredient delivery system comprises an active ingredient reservoir. The active ingredient reservoir is fluidically connected to the conveying device. In the active ingredient reservoir, the active ingredient to be administered to the patient is provided in solid and/or powder and/or liquid form. According to the invention, a delivery device is also fluidically connected to the conveying device. The delivery device is used for intradermal delivery of a mixture of active ingredient and extracted interstitial liquid or for delivery of the interstitial liquid, which is enriched with active ingredient, to the patient. The delivery device preferably comprises microneedles designed in particular as hollow needles.

The intradermal drug delivery system according to the invention thus makes it possible to administer a clearly defined amount of active ingredient to the patient. Here, it is preferred that in the active ingredient reservoir the active ingredient is in concentrated form and is diluted by the interstitial liquid or is dissolved by the interstitial liquid, respectively.

In a particularly preferred further embodiment of the invention, the extraction device in particular has a plurality of microneedles. Preferably, one or more microneedle arrays are provided which, in a preferred embodiment, have at least 4 needles/cm², preferably at least 50 needles/cm², and particularly preferred at least 100 needles/cm² per array.

In particular, if the extraction device has several microneedle arrays, it is preferred that these are arranged parallel to each other and are each connected to the conveying device via a separate channel. Furthermore, it is possible for several extraction devices to be arranged at the same distance from the conveying device, in particular circular, around the conveying device. Providing several microneedle arrays in particular has the advantage that these can each be connected to channels with a small cross-section and in this respect a very small intradermal active ingredient system can be realized. Providing several microneedle arrays also has the advantage that a sufficient liquid supply is always ensured. The liquid adapted to be drawn in can be realized in particular in connection with a liquid conveying device with a pump with increased stroke and/or increased number of strokes.

Preferably, the conveying device has at lest one pump. The pump can be a diaphragm pump or the like, which in particular has valves designed in such a way that liquid can only be conveyed in one direction. In particular, diaphragms are provided respectively at the inlet and the outlet of the pump. This ensures in particular that mixing with the active ingredient preferably only takes place within the pump or a space provided within the pump, respectively. In particular, it is avoided that the active ingredient is not already diluted without active pumping or dosing. Furthermore, providing diaphragms on the microneedle arrays has the advantage that the channels are always constantly filled with liquid or an active ingredient solution and backflow into the skin or a chamber arranged in the pump is avoided. For example, the pump could be controlled by a piezo activator that causes a pressure change on a pump chamber to activate the pumping process. It is also conceivable that the pumping is done by manually generated pressure on a corresponding chamber.

In a particularly preferred embodiment of the invention, an active ingredient reservoir containing liquid active ingredient is connected to an inlet of the conveying device. Preferably, the extraction device is also connected to the inlet of the conveying device. In particular, the connection is made with the inlet of a pump. The pump can have one or more inlets, although providing a single inlet is preferred to miniaturize the active ingredient delivery system.

In this preferred embodiment of the invention, liquid active ingredient and interstitial liquid can already be drawn in simultaneously by the conveying device. Here, the two liquids are preferably mixed. Optionally, a mixing chamber can be provided, in particular within the conveying device, in which the two liquids are mixed. The chamber may in particular be a chamber of the pump, particularly of the diaphragm pump. The two mixed liquids are then preferably delivered with the aid of the conveying device to the dermis of the skin via a delivery device connected to the conveying device.

In a further preferred embodiment, the active ingredient reservoir is connected to an outlet of the conveying device, in particular an outlet of a pump of the conveying device. Here, a liquid, solid or powdered active ingredient may be provided in the active ingredient reservoir. With the aid of the conveying device, in this preferred embodiment, the interstitial liquid is preferably drawn in via the extraction device and the interstitial liquid is conveyed into the active ingredient reservoir. In this embodiment, the active ingredient reservoir can simultaneously serve as a mixing chamber, or mixing or dissolving of the active ingredient takes place in the active ingredient reservoir, respectively.

In a preferred embodiment, the conveying device can have several pumps, in particular two pumps. In this embodiment, it is possible that the active ingredient reservoir is arranged between the two pumps. The pump arranged upstream of the active ingredient reservoir in the direction of flow is hereby used to draw in the interstitial liquid and then pump the interstitial liquid into the active ingredient reservoir. The pump arranged downstream of the reservoir in the direction of flow draws in the interstitial liquid enriched with active ingredient and conveys it to the delivery device.

In a preferred embodiment, the delivery device, which in particular has a plurality of hollow needles, is connected directly or indirectly to the conveying device, in particular via one or more channels.

In a further preferred embodiment, the delivery device can be connected directly or indirectly to the active ingredient chamber or a mixing chamber, in particular via channels. In this embodiment, it is preferred that the pump conveys drawn-in interstitial fluid through the active ingredient reservoir and the mixing chamber. Here, the interstitial liquid absorbs active ingredient so that interstitial liquid enriched with active ingredient reaches the delivery device.

In a further preferred embodiment of the intradermal active ingredient delivery system according to the invention, the active ingredient reservoir or an additional active ingredient reservoir is integrated into the delivery device. Mixing the interstitial fluid with the active ingredient disposed in such an active ingredient reservoir thus takes place immediately before delivery to the patient. In particular, the active ingredient may be disposed directly in the hollow needles.

Optionally, the active ingredient system may include a mixing chamber. Here, a mixing element such as a piezo actuator or the like may act on the mixing chamber to cause good mixing of the active agent with the interstitial liquid. Here, a reliable dissolution of the active ingredient can also be ensured for an active ingredient in solid or powder form. Depending on the embodiment, the mixing chamber may be omitted or, for example, integrated into the conveying device, the active ingredient reservoir and/or the delivery device.

The active ingredient delivery system according to the invention has a size such that the entire system can be reliably arranged on the patient's skin in a simple manner. In addition to the small design on a contact surface with the skin of only a few cm², active ingredient delivery systems could also be realized, for example in the form of a wristband enclosing the arm. Particularly important for realizing the active ingredient delivery system according to the invention is the flexibility of the system and the penetration depth of the needles. To realize the required flexibility, the system is always so thin that it remains permanently in contact with the entire surface of the skin, even during movements, and on the other hand the patient's freedom of movement is only slightly restricted. The needles have a length that they penetrate only into the dermis of the skin, so that a possibly painful contact with nerves is avoided.

### BRIEF DESCRIPTION OF THE DRAWINGS

In the following, the invention is described in more detail by means of preferred embodiments with reference to the accompanying drawings.

In the drawings:

FIGS. 1-4 show different preferred embodiments of the intradermal active ingredient delivery system according to the invention,

FIG. 5 shows a schematic highly simplified sectional view of an extraction device or a delivery device, and

FIG. 6 shows a schematic highly simplified illustration of a conveying device in the form of a diaphragm pump.

### DESCRIPTION OF THE INVENTION

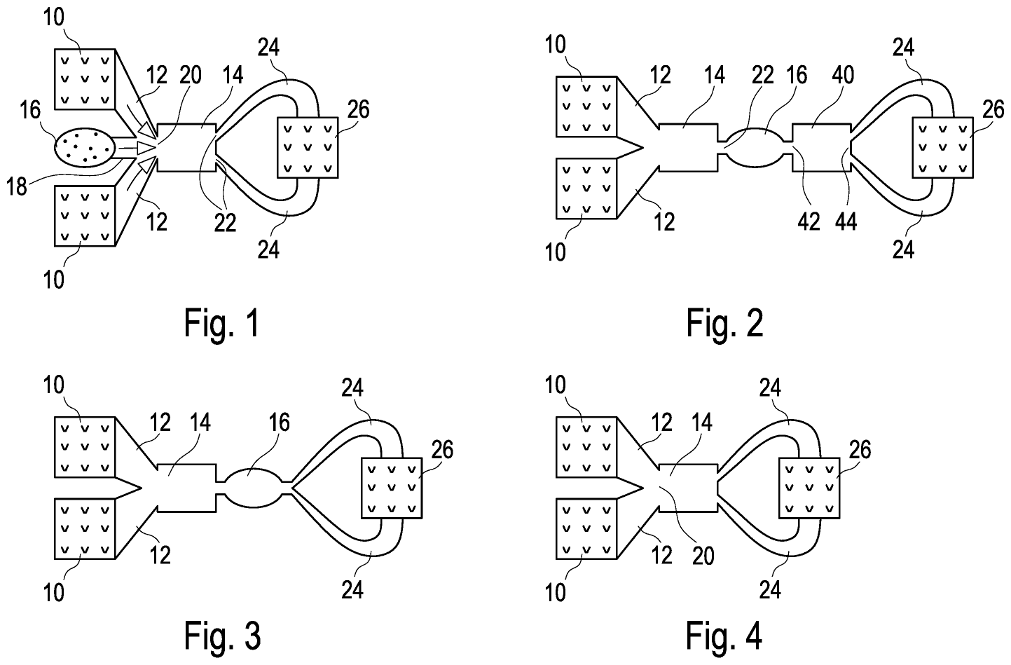

In the various embodiments of the intradermal active ingredient delivery system according to the invention shown in FIGS. 1-4, identical and similar components are marked with the same reference numerals.

The embodiment shown in FIG. 1 comprises an extraction device 10 having two microarrays with a plurality of hollow needles. The two microarrays 10 are each connected to a pump or a conveying device 14 via channels 12. An active ingredient reservoir 16, in which liquid active ingredient is stored, is also connected to pump 14 via a channel 18. In the illustrated exemplary embodiment, all three channels 12, 18 are merged upstream of pump 14 and connected to a single pump inlet 20.

In the illustrated exemplary embodiment, pump 14 comprises two pump outlets 22, each connected to a channel 24. Alternatively, pump 14 may also have an outlet which is then also connected to the two channels 24. The channels 24 are connected to a delivery device 26. The delivery device 26 is a microarray with a plurality of hollow needles.

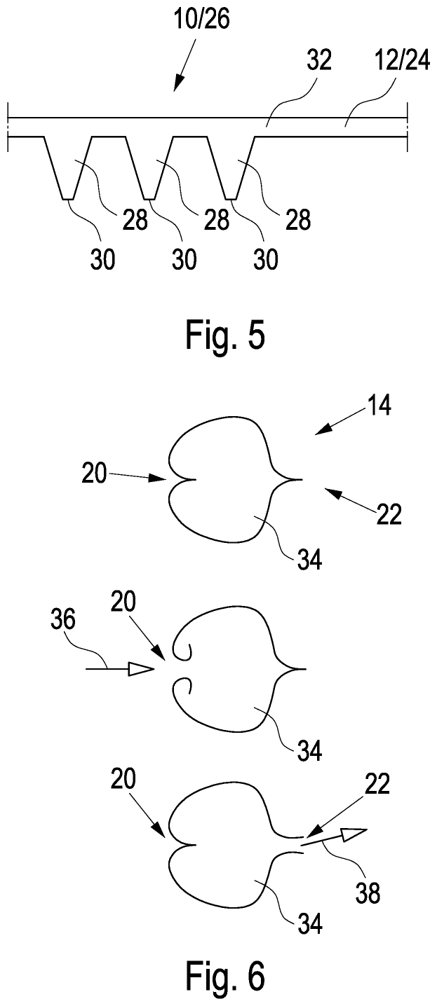

FIG. 5 schematically shows a section of a microarray, which can be a microarray 10 of the extraction device or a microarray 26 of the delivery device. The microarray 10, 26 comprises a plurality of hollow needles 28 having an opening 30 at the tip. Interstitial liquid can be drawn in through opening 30 if the microarray is an extraction device. If the microarray is a delivery device, the interstitial liquid enriched with active ingredient is delivered to the patient through the openings 30. The hollow needles 28 are connected to a channel 32 on the side opposite the openings 30. The channel 32 preferably extends over the entire surface of the microarray and is connected to either channel 12 or channel 24.

The intradermal active ingredient delivery system shown in FIG. 1 allows interstitial fluid to be drawn in by the pump through the channels 12 via the hollow needles of the microarrays 10. At the same time, liquid active ingredient is drawn in from the active ingredient reservoir 16. Within pump 14 or already in the area where the channels 12 and 18 are merged, the two liquids are mixed. After closing pump inlet 20, pump 14 can be used to convey the fluid mixture through the two outlets 22 and the channels 24 to the microneedles 30 of delivery system 26 and inject it into the patient.

For example, pump 14 may be a diaphragm pump shown schematically in FIG. 6. In the state of pump 14 shown in FIG. 6, both outlet 22 and inlet 20 are closed. The pump 14 is in an unloaded state. For example, force can be applied to a pump cavity 34 by a piezo element not shown. When negative pressure or vacuum is created in pump cavity 34, inlet 20 opens to allow liquid to flow into cavity 34 in the direction of an arrow 36. To convey the liquid out of pump cavity 34, pressure is again applied to cavity 34, for example by means of a piezo element. This closes inlet 20 according to the lower illustration in FIG. 6. At the same time, outlet 22 opens so that liquid flows in the direction of an arrow 38.

According to another embodiment shown in FIG. 2, the two microarrays 10 of the extraction device are again connected to a first pump 14 via channels 12. In this exemplary embodiment, outlet 22 of the first pump 14 is connected to active ingredient reservoir 16. The active ingredient reservoir 16 is then connected in the direction of flow, i.e. to the right in FIG. 2, to a second pump 40 via its inlet 42. An outlet 44 of the second pump 40 is connected to delivery device 26 via the channels 24.

In the exemplary embodiment shown in FIG. 2, the first pump 14 thus draws in interstitial liquid via extraction device 10 and conveys it into active ingredient reservoir 16. A liquid, solid or powdered active ingredient is provided in active ingredient reservoir 16. Thus, in the exemplary embodiment shown in FIG. 2, active ingredient reservoir 16 also serves as a mixing chamber. The second pump 40 draws in the liquid mixture from active ingredient reservoir 16 and conveys it to delivery device 26 via the channels 24.

The exemplary embodiment shown in FIG. 3 substantially corresponds to the exemplary embodiment shown in FIG. 2, with the difference that no second pump is provided. The interstitial liquid drawn in by pump 14 is thus pumped by pump 14 through active ingredient reservoir 16, wherein the interstitial liquid absorbs active ingredient. The pump 14 then continues to convey the liquid mixture directly through the channels 24 to delivery device 26.

The embodiment shown in FIG. 4 substantially corresponds to the embodiment shown in FIG. 1, wherein no active ingredient reservoir 16 is connected to pump inlet 20.

Rather, in this exemplary embodiment, the active ingredient reservoir is integrated into extraction device 26. For example, the active ingredient is provided in the hollow needles 30 and/or in channel region 32 (FIG. 5) and is absorbed by the interstitial liquid immediately prior to injection.

1. An intradermal active ingredient delivery system comprising
an extraction device for extracting interstitial liquid,
a conveying device fluidically connected to the extraction device for conveying liquid,
an active ingredient reservoir fluidically connected to the conveying device, and
a delivery device fluidically connected to the conveying device for intradermal delivery of the interstitial liquid comprising an active ingredient.

2. The intradermal active ingredient delivery system according to claim 1, wherein the extraction device comprises microneedles, in particular a microarray having at least 4 needles/cm², in particular at least 50 needles/cm², and particularly preferred at least 100 needles/cm².

3. The intradermal active ingredient delivery system according to claim 2, wherein the extraction device comprises two microarrays each having in particular at least 4 needles/cm², in particular at least 50 needles/cm², and particularly preferred at least 100 needles/cm².

4. The intradermal active ingredient delivery system according to claim 1, wherein the conveying device comprises at least one pump.

5. The intradermal active ingredient delivery system according to claim 1, wherein an active ingredient in solid, liquid or powder form is stored in the active ingredient reservoir.

6. The intradermal active ingredient delivery system according to claim 1, wherein the active ingredient reservoir containing the liquid active ingredient and the extraction device are connected to an inlet of the conveying device, in particular to a pump inlet.

7. The intradermal active ingredient delivery system according to claim 6, wherein the active ingredient reservoir and the extraction device are connected to the inlet of the conveying device, in particular to the pump inlet, in such a way that the active substance and the initial liquid are drawn in simultaneously.

8. The intradermal active ingredient delivery system according to claim 1, wherein the active ingredient reservoir is connected to an outlet of the conveying device, in particular to a pump outlet.

9. The intradermal active ingredient delivery system according to claim 1, wherein the conveying device comprises two pumps, and that the active ingredient reservoir is arranged between the two pumps.

10. The intradermal active ingredient delivery system according to claim 1, wherein the delivery device comprises microneedles, in particular a microarray having at least 4 needles/cm², in particular at least 50 needles/cm², and particularly preferred at least 100 needles/cm².

11. The intradermal active ingredient delivery system according to claim 1, wherein the delivery device is connected to the outlet of the conveying device, in particular to the pump outlet.

12. The intradermal active ingredient delivery system according to claim 1, wherein the active ingredient reservoir is integrated into the delivery device, wherein the active ingredient is preferably provided in the needles of the delivery device.

13. The intradermal active ingredient delivery system according to claim 1, characterized by a mixing chamber for mixing interstitial liquid and active ingredient.

14. The intradermal active ingredient delivery system according to claim 13, wherein the active ingredient reservoir forms the mixing chamber.

15. The intradermal active ingredient delivery system according to claim 13, wherein the mixing chamber is integrated into the conveying device.
